(19) United States
(12) Patent Application Publication
(10) Pub. No.: US 2017/0209268 A1
(43) Pub. Date: Jul. 27, 2017

(54) SYSTEMS AND METHODS FOR REPOSITIONING A FULLY DEPLOYED VALVE ASSEMBLY

(71) Applicant: Medtronic, Inc., Minneapolis, MN (US)

(72) Inventors: Kieran Cunningham, Ballybrit (IE); Marian Lally, Ballybrit (IE)

(21) Appl. No.: 15/008,019

(22) Filed: Jan. 27, 2016

(51) Int. Cl. A61F 2/24; A61F 2/95

(52) U.S. Cl. CPC A61F 2/2439; A61F 2/243; A61F 2/2418; A61F 2/95; A61F 2002/9534; A61F 2230/0069

(57) ABSTRACT

A valve assembly includes a frame, a prosthetic valve coupled to the frame, and a repositioning wire coupled to the frame. When pulled, the repositioning wire is configured to radially compress the valve assembly from a radially expanded fully deployed configuration to a radially compressed repositioning configuration. The repositioning wire includes a first end coupled to the frame and the repositioning wire extends around at least a portion of the circumference of the frame to a second end. The second end of the repositioning wire may include a lasso.

### FIELD OF THE INVENTION

The present disclosure relates to devices, systems, and methods for repositioning a fully deployed valve assembly.

### BACKGROUND

Heart valves are sometimes damaged by disease or by aging, resulting in problems with the proper functioning of the valve. Heart valve replacement has become a routine surgical procedure for patients suffering from valve dysfunctions. Traditional open surgery inflicts significant patient trauma and discomfort, requires extensive recuperation times, and may result in life-threatening complications.

To address these concerns, minimally invasive techniques, such as transcatheter valve implantation techniques, have been developed to deliver and deploy valve prostheses. In such methods, the valve prosthesis or valve assembly generally includes a frame and a prosthetic valve, and is radially compressed for delivery in a catheter and then advanced to the location of a native valve, where the valve assembly is deployed by radial expansion. The catheter may be advanced, for example through an opening in the native vasculature remote from the native valve, such as the femoral artery, and advanced through the vasculature to the native valve. In other techniques, the catheter is advanced through an opening in the heart to the location of the native valve, such as transapical or transatrially, or through an opening in the ascending aorta.

In some patients, the valve assembly may not perform as desired following implantation. For example, due to the position of the valve assembly, the valve assembly may not properly seal with the native valve and/or walls surrounding the native valve. This may result in paravalvular leakage (PVL), and other post surgical complications. Further, the valve assembly may not function properly due to the position of the valve assembly at the native valve. However, once a valve assembly is fully deployed and released from the delivery device, there is no easy way to reposition to the valve assembly to a new location.

Accordingly, there is a need for a valve assembly, system and method of repositioning a fully deployed valve assembly.

### SUMMARY OF INVENTION

Embodiments hereof relate to a valve assembly including a frame, a prosthetic valve, and a repositioning wire. The frame defines a central passage. The prosthetic valve is coupled to the frame and disposed in the central passage of the frame. The repositioning wire is coupled to the frame. The repositioning wire is configured such that with the valve assembly in a radially expanded fully deployed configuration, pulling the repositioning wire radially compresses the valve assembly from the radially expanded fully deployed configuration to a radially compressed repositioning configuration.

Embodiments hereof also relate to a valve assembly repositioning system for repositioning a valve assembly that is in a radially expanded fully deployed configuration. The valve assembly repositioning system includes the valve assembly and a snare device. The valve assembly includes a frame that defines a central passage, a prosthetic valve coupled to the frame, and a repositioning wire coupled to the frame. The snare device is configured to snare and pull the repositioning wire to radially compress the valve assembly from the radially expanded fully deployed configuration to a radially compressed repositioning configuration. The snare device is also configured to move the valve assembly when the valve assembly is in the radially compressed repositioning configuration.

Embodiments hereof also relate to a method of repositioning a valve assembly having a frame, a prosthetic valve coupled to the frame, and a repositioning wire coupled to the frame. The method includes advancing a snare device to a location of the valve assembly with the valve assembly in a radially expanded fully deployed configuration at a first location adjacent a native valve. The snare device snares the repositioning wire. The snare device is manipulated such that the repositioning wire is pulled to radially compress the valve assembly from the radially expanded fully deployed configuration to a radially compressed repositioning configuration. The valve assembly is moved from the first location to a second location adjacent the native valve by manipulation of the snare device. The repositioning wire is released from the snare device and the valve assembly radially expands from the radially compressed repositioning configuration back to the radially expanded fully deployed configuration.

### BRIEF DESCRIPTION OF DRAWINGS

FIG. 1A is a side perspective schematic illustration of a valve assembly according to an embodiment hereof with the valve assembly in a radially expanded fully deployed configuration.

FIG. 1B an end view schematic illustration of the valve assembly of FIG. 1A.

FIG. 2A is a side view schematic illustration of the valve assembly of FIG. 1A in a radially compressed repositioning configuration.

FIG. 2B is an end view schematic illustration of the valve assembly of FIG. 1A in a radially compressed repositioning configuration.

FIG. 3 is a side perspective schematic illustration of a valve assembly repositioning system according to an embodiment hereof, with the valve assembly of FIG. 1A.

FIGS. 4A-4D are a series of side illustrations of a snare device grasping and pulling a repositioning wire.

FIG. 5A is a side perspective schematic illustration of a valve assembly according to another embodiment hereof, wherein the repositioning wire includes a lasso.

FIG. 5B is an end view perspective schematic illustration of the valve assembly of FIG. 5A.

FIG. 6 is a side perspective schematic illustration of a valve assembly repositioning system according to an embodiment hereof, with the valve assembly of FIG. 5A.

FIGS. 7A-7E are a series of close up side illustrations of snare device snaring and pulling the lasso of the repositioning wire.

FIG. 8A is a side perspective schematic illustration of the valve assembly of FIG. 5A, wherein the valve assembly includes a plurality of repositioning wires with lassos.

FIG. 8B is an end view perspective schematic illustration of the valve assembly of FIG. 8A.

FIG. 9 is a side perspective schematic illustration of a valve assembly repositioning system according to an embodiment hereof, with the valve assembly of FIG. 8.

FIGS. 10A-10D are a series of side schematic illustrations of a snare device snaring and pulling the lassos of the repositioning wires of the valve assembly of FIG. 8.

FIGS. 11-16 are schematic illustrations of an embodiment of a method of repositioning a fully deployed valve assembly.

FIG. 11 is a schematic illustration of the valve assembly of FIG. 1A in a radially expanded fully deployed configuration and disposed at a first location adjacent a native valve.

FIG. 12 is a schematic illustration a step in the method of repositioning the valve assembly, wherein the snare device has grasped the repositioning wire.

FIG. 13 is a schematic illustration of a step in the method of repositioning the valve assembly, wherein the snare device is pulling the repositioning wire and the valve assembly is collapsing to the radially collapsed repositioning configuration.

FIG. 14 is a schematic illustration of a step in the method of repositioning the valve assembly, wherein the valve assembly is in the radially collapsed repositioning configuration and is being moved to a second location adjacent the native valve.

FIG. 15 is a schematic illustration of a step in the method of repositioning the valve assembly, wherein the snare device is releasing the repositioning wire and the valve assembly is expanding to the radially expanded fully deployed configuration at the second location.

FIG. 16 is a schematic illustration of a step in the method of repositioning the valve assembly, wherein the snare device has released the valve assembly such that the valve assembly is in the radially expanded fully deployed configuration at the second location.

FIGS. 17-22 are schematic illustrations of another embodiment of a method of repositioning a fully deployed valve assembly.

FIG. 17 is a schematic illustration of the valve assembly of FIG. 8A in a radially expanded fully deployed configuration and disposed at a first location adjacent a native valve.

FIG. 18 is a schematic illustration of a step in the method of repositioning the valve assembly, wherein the snare device has snared the lassos of the repositioning wires.

FIG. 19 is a schematic illustration of a step in the method of repositioning the valve assembly, wherein the snare device is pulling the repositioning wires to collapse the valve assembly to the radially collapsed repositioning configuration.

FIG. 20 is a schematic illustration of a step in the method of repositioning the valve assembly, wherein the valve assembly is in the radially collapsed repositioning configuration and is being moved to a second location adjacent the native valve.

FIG. 21 is a schematic illustration of a step in the method of repositioning the valve assembly, wherein the snare device is releasing the repositioning wire and the valve assembly is expanding to the radially expanded fully deployed configuration at the second location.

FIG. 22 is a schematic illustration of a step in the method of repositioning the valve assembly, wherein the snare device has released the valve assembly such that the valve assembly is in the radially expanded fully deployed configuration at the second location.

### DETAILED DESCRIPTION

Specific embodiments of the present invention are now described with reference to the figures, wherein like reference numbers indicate identical or functionally similar elements. The terms “distal” and “proximal”, when used in the following description to refer to a catheter or delivery device, are with respect to a position or direction relative to the treating clinician. Thus, “distal” and “distally” refer to positions distant from, or in a direction away from, the clinician and “proximal” and “proximally” refer to positions near, or in a direction toward, the clinician. When the terms “distal” and “proximal” are used in the following description to refer to a device implanted into a native artery, such as a valve assembly, they are used with reference to the direction of blood flow from the heart. Thus “distal” and “distally” refer to positions in a downstream direction with respect to the direction of blood flow and “proximal” and “proximally” refer to positions in an upstream direction with respect to the direction of blood flow.

The following detailed description is merely exemplary in nature and is not intended to limit the invention or the application and uses of the invention. Although the description of the invention is in the context of a transcatheter aortic valve repositioning system, the invention may also be used in other body passageways where it is deemed useful. Furthermore, there is no intention to be bound by any expressed or implied theory presented in the preceding technical field, background, brief summary, or the following detailed description.

As used herein the terms “fully deployed”, “fully deployed configuration”, and “radially expanded fully deployed configuration” mean that the device, such as a valve assembly or frame, that is described using these terms has been deployed at a site within the body, has been radially expanded (such as by balloon expansion or self-expansion), and has been released from the delivery device. Thus, for example, a valve assembly wherein a portion of the valve assembly has been radially expanded but a portion of the valve assembly is either not radially expanded or is still attached to the delivery device, is not considered “fully deployed”, in a “fully deployed configuration”, or in a “radially expanded fully deployed configuration”.

In general terms, the valve assembly of the present disclosure includes a frame, a prosthetic valve, and at least one repositioning wire. The valve assembly has a radially expanded fully deployed configuration that is collapsible to a radially compressed repositioning configuration for repositioning the valve assembly after the valve assembly has been fully deployed adjacent a native valve. The valve assembly also has a radially compressed delivery configuration (not shown), which may be a smaller diameter than the radially compressed repositioning configuration.

The frame of the valve assembly is a generally tubular configuration having a proximal end, a distal end, and a lumen therebetween. The frame is a stent structure as is known in the art, as described in more detail below. The frame may be self expanding or may be balloon expandable. The frame may comprise a number of strut or wire portions arranged relative to each other to provide a desired compressibility, strength, and leaflet attachment zone(s). The frame is a generally tubular support structure, and leaflets are secured to the frame to provide a stented prosthetic valve.

The prosthetic valve of the valve assembly may be attached to the frame. The prosthetic valve may also include a skirt affixed to the frame. The prosthetic valve may include a plurality of prosthetic valve leaflets, which may be attached along their bases to the skirt, for example, using sutures or a suitable biocompatible adhesive, or may be attached to the skirt or frame in other ways known to those skilled in the art. Adjoining pairs of leaflets may be attached to one another at their lateral ends to form commissures with free edges of the leaflets forming coaptation edges that meet in an area of coaptation. The prosthetic valve leaflets may be formed from a variety of materials, such as autologous tissue, xenograph material, or synthetics as are known in the art. The leaflets may be provided as a homogenous, biological valve structure, such as a porcine, bovine, or equine valve. Alternatively, the leaflets can be provided independent of one another (e.g., bovine or equine pericardial leaflets) and subsequently assembled to the support structure of the frame. In another alternative, the frame and leaflets may be fabricated at the same time, such as may be accomplished using high strength nano-manufactured NiTi films of the type produced at Advanced Bio Prosthetic Surfaces Ltd. (ABPS) of San Antonio, Tex., for example.

The frame and prosthetic valve of the valve assembly may be similar to the Medtronic CoreValve® transcatheter aortic valve replacement valve prosthesis and as described in U.S. Patent Application Publication No. 2011/0172765 to Nguyen et al., which is incorporated by reference herein in its entirety. However, those skilled in the art would recognize that any suitable valve prosthesis may be used in the present embodiment as the frame and prosthetic valve of the valve assembly. For example, and not by way of limitation, the combination of a frame and prosthetic valve of the valve assembly may assume a variety of other configurations that differ from those shown and described, including any known prosthetic heart valve design. In various embodiments, the frame and the prosthetic valve may utilize certain features of known expandable prosthetic heart valve configurations, whether balloon expandable, self-expanding, or unfurling (as described, for example, in U.S. Pat. Nos. 3,671,979; 4,056,854; 4,994,077; 5,332,402; 5,370,685; 5,397,351; 5,554,185; 5,855,601; and 6,168,614; U.S. Patent Application Publication No. 2004/0034411; Bonhoeffer P., et al., “Percutaneous Insertion of the Pulmonary Valve”, Pediatric Cardiology, 2002; 39:1664-1669; Anderson H R, et al., “Transluminal Implantation of Artificial Heart Valves”, EUR Heart J., 1992; 13:704-708; Anderson, J. R., et al., “Transluminal Catheter Implantation of New Expandable Artificial Cardiac Valve”, EUR Heart J., 1990, 11: (Suppl) 224a; Hilbert S. L., “Evaluation of Explanted Polyurethane Trileaflet Cardiac Valve Prosthesis”, J Thorac Cardiovascular Surgery, 1989; 94:419-29; Block P C, “Clinical and Hemodynamic Follow-Up After Percutaneous Aortic Valvuloplasty in the Elderly”, The American Journal of Cardiology, Vol. 62, Oct. 1, 1998; Boudjemline, Y., “Steps Toward Percutaneous Aortic Valve Replacement”, Circulation, 2002; 105:775-558; Bonhoeffer, P., “Transcatheter Implantation of a Bovine Valve in Pulmonary Position, a Lamb Study”, Circulation, 2000: 102:813-816; Boudjemline, Y., “Percutaneous Implantation of a Valve in the Descending Aorta In Lambs”, EUR Heart J, 2002; 23:1045-1049; Kulkinski, D., “Future Horizons in Surgical Aortic Valve Replacement: Lessons Learned During the Early Stages of Developing a Transluminal Implantation Technique”, ASAIO J, 2004; 50:364-68; the teachings of which are all incorporated herein by reference).

The valve assembly of the present disclosure adds at least one repositioning wire. The term “wire” as used herein means an elongated element or filament or group of elongated elements or filaments and is not limited to a particular cross-sectional shape or material, unless so specified. The repositioning wire of the present disclosure includes a first end coupled to the frame. The repositioning wire extends from the first end around a circumference of the frame to a second end disposed opposite the first end. The first end of the repositioning wire may be coupled to the frame of the valve assembly by methods such as, but not limited to laser or ultrasonic welding, adhesives, tying, or other methods suitable for the purposes disclosed herein. The repositioning wire wraps around at least a portion of the circumference of the valve assembly. In an embodiment, with the valve assembly is in the radially expanded fully deployed configuration, the repositioning wire wraps around at least 75% of the circumference of the frame at the location of the repositioning wire. The repositioning wire may be woven through the open spaces of the frame of the valve assembly, above some frame members and below others. The repositioning wire is configured such that when the second end of the repositioning wire is pulled, the valve assembly is compressed from the radially expanded fully deployed configuration to the radially compressed repositioning configuration. The repositioning wire may be constructed of materials such as, but not limited to stainless steel, Nitinol, nylon, polybutester, polypropylene, silk, and polyester or other materials suitable for the purposes described herein.

The present disclosure also discloses a valve assembly repositioning system, which includes a valve assembly, as described above, and a snare device. The snare device is an elongate member configured to snare and to pull the repositioning wire of the valve assembly such that the valve assembly is compressed from the radially expanded fully deployed configuration to the radially compressed repositioning configuration. The snare device is also configured to move the valve assembly from a first location adjacent a native valve to a second location adjacent a native valve when the valve assembly is in the radially compressed repositioning configuration.

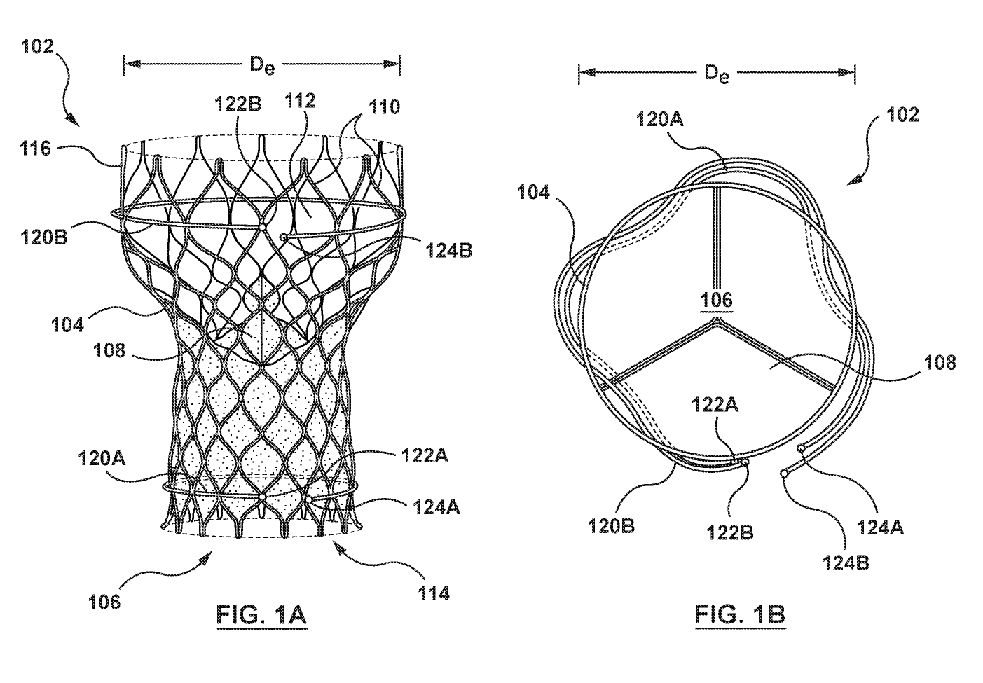

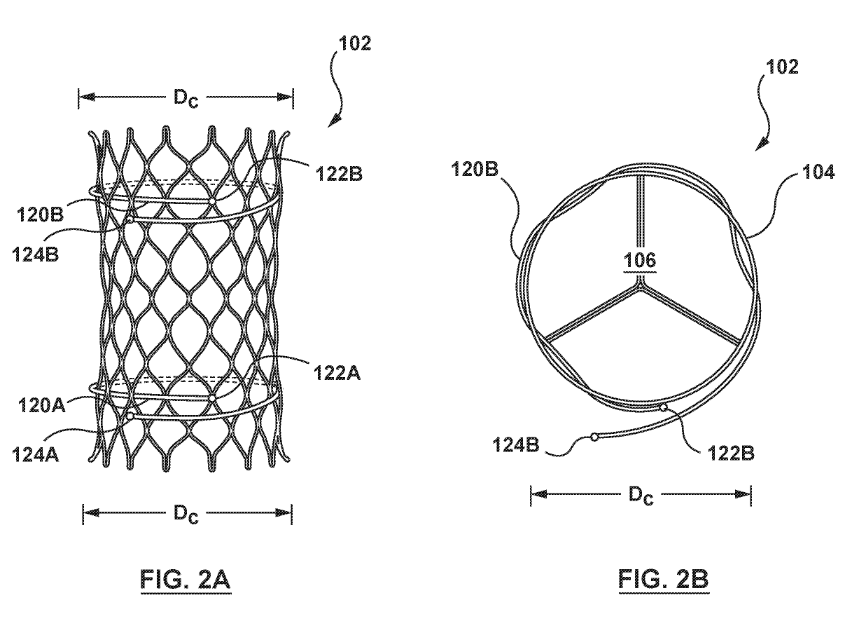

With the above understanding in mind, an embodiment of a valve assembly 102 according to the present disclosure is shown in FIGS. 1A-2B. FIG. 1A illustrates valve assembly 102 in a radially expanded fully deployed configuration. Valve assembly 102 includes a frame 104, a prosthetic valve 108, and a repositioning wire 120, as described above.

Frame 104 is of a generally tubular configuration including a first end 104 and a second end 106, and defines a central passage 106 therethrough. Frame 104 is a support structure that comprises a number of wire members 110 arranged relative to each other to create open spaces 112. Prosthetic valve 108 is coupled to frame 104 and disposed within central passage 106 of frame 104.

Valve assembly 102 further includes a first repositioning wire 120A and a second repositioning wire 120B. In the embodiment shown, first repositioning wire 120A is disposed adjacent first end 114 (inflow end) of frame 104 and second repositioning wire 120B is disposed adjacent second end 116 (outflow end) of frame 104. Although FIGS. 1A-2B show two repositioning wires, more or fewer repositioning wires may be utilized. For example, and not by way of limitation, a single reposition wire may be utilized adjacent the first end, second end, or middle portion of frame 104. Alternatively, and also not by way of limitation, a third repositioning wire may be added between the first and second repositioning wires. As explained above, first repositioning wire 120A includes a first end 122A coupled to frame 104. First repositioning wire 120A wraps around a circumference of frame 104 to a second end 124A, as shown in FIG. 1A. Similarly, second repositioning wire 120B includes a first end 122B coupled to frame 104. Second repositioning wire 120B wraps around a circumference of frame 104 to a second end 124B, as shown in FIG. 1A. In an embodiment, with valve assembly 102 in the radially expanded fully deployed configuration, each of first and second repositioning wires 120A, 120B wraps around the circumference of frame 104 at least 75% of the circumference of frame 104 at the location of each respective repositioning wire. As previously described, each repositioning wire 120A, 120B may be woven through open spaces 112 of frame 104, above some frame members 110 and below others.

Each repositioning wire 120A, 120B is configured such that when respective second end 124A, 124B of each repositioning wire 120A, 120B is pulled, frame 104 of valve assembly 102 is compressed from the radially expanded fully deployed configuration to a radially compressed repositioning configuration, thereby also compressing valve assembly 102 from the radially expanded fully deployed configuration to the radially compressed repositioning configuration. As shown in FIGS. 1A-2B, valve assembly 102 has an outer diameter Dₑ when in the radially expanded fully deployed configuration, as shown in FIGS. 1A-1B which is greater than an outer diameter D_{c} when in the radially compressed repositioning configuration, as shown in FIGS. 2A-2B. Outer diameter D_{c} may be in the range of 40% to 80% of outer diameter Dₑ.

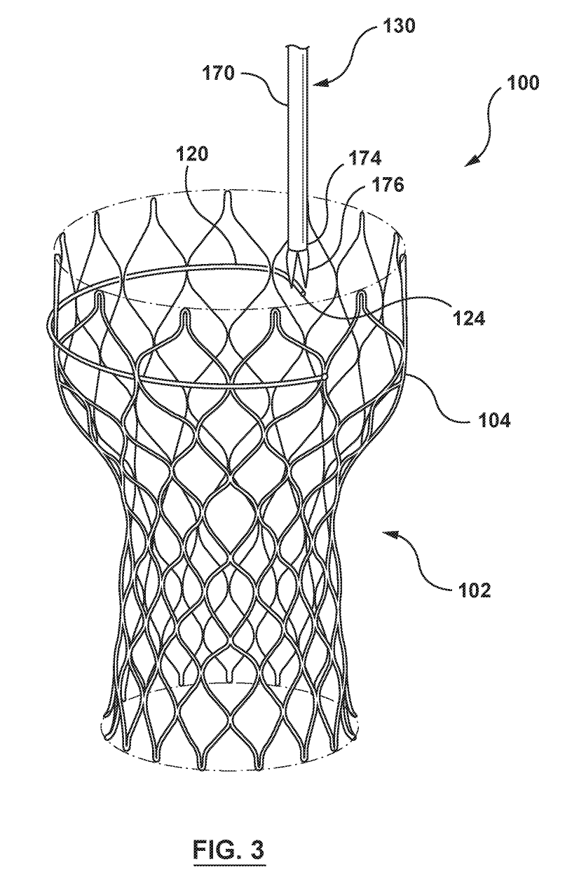

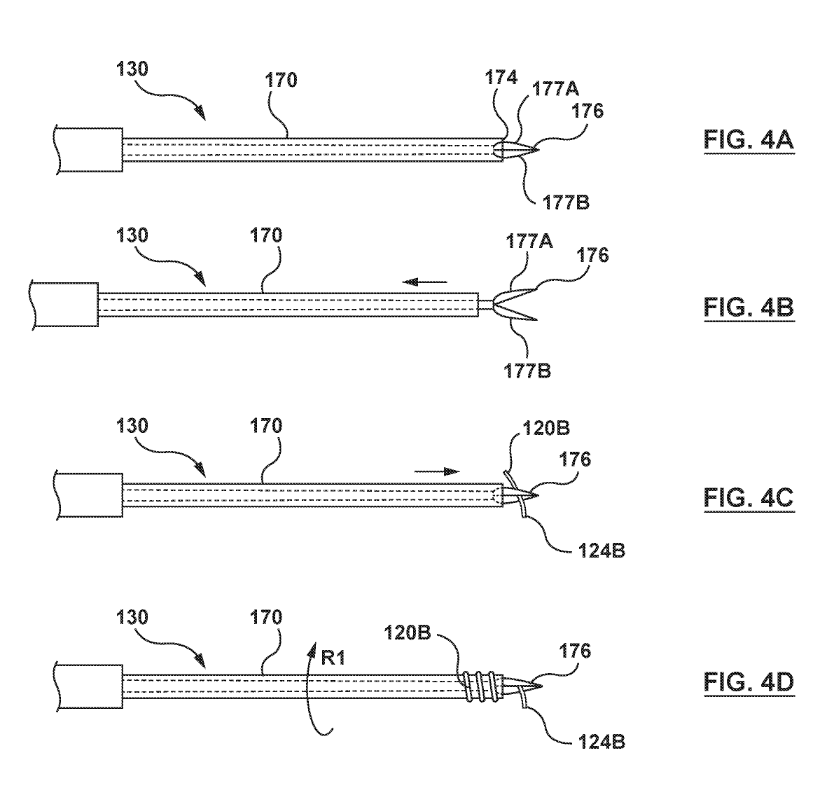

FIG. 3 shows an embodiment of a valve assembly repositioning system 100 including valve assembly 102 and a snare device 130. Snare device 130, shown in more detail in FIGS. 4A-4D, is an elongated device including a shaft 170 configured for delivery through the vasculature, and a clasping mechanism 176 disposed at a distal end 174 of shaft 170 of snare device 130. More particularly, clasping mechanism 176 is user actuated from a location at a proximal end (not shown) of snare device 130. Clasping mechanism 176 is configured to grasp and hold second end 124B of repositioning wire 120B, as shown in FIG. 3. Although snare device 130 in FIG. 3 is shown grasping repositioning wire 120B, snare device 130 could instead grasp repositioning wire 120A, or snare device 130 may include a plurality of clasping mechanisms, one for each repositioning wire, or a plurality of snare devices 130 could be used, one for each repositioning wire. Snare device 130 is further configured to pull repositioning wire 120B such that valve assembly 102 is compressed from the radially expanded fully deployed configuration to the radially compressed repositioning configuration. In the embodiment shown in FIGS. 3 and 4A-4D, repositioning wire 120B is pulled by rotating snare device 130 in a direction R1 such that repositioning wire 120B wraps circumferentially around shaft 170 of snare device 130, as shown in FIGS. 4A-4D. To release repositioning wire 120B, snare device 130 is rotated in a direction (not shown) opposite direction R1.

Snare device 130 is also configured to move valve assembly 102 longitudinally within the native vessel or valve when valve assembly 102 is in the radially compressed repositioning configuration. To move valve assembly 102 distally or proximally, the user pushes or pulls snare device 130, respectively. Stated another way, when snare device 130 has grasped and pulled repositioning wire 120 and valve assembly 102 is in the radially compressed repositioning configuration, moving snare device 130 distally moves valve assembly 102 distally (i.e., away from the clinician), and moving snare device 130 proximally moves valve assembly 102 proximally (i.e., towards the clinician).

Clasping mechanism 176 shown in FIGS. 3-4D is shown including a pair of jaws 177A, 177B. However, any clasping mechanism suitable to grasp and hold a repositioning wire may be utilized. In one embodiment, jaws 177A, 177B are displaceable towards and away from one another and are formed from a resilient material. In an embodiment, jaws 177A, 177B are biased into a normally open configuration, as shown in FIG. 4B. For delivery to the location of valve assembly 102, shaft 170 is extended at least partially over clasping mechanism 176 to maintain jaws 177A, 177B in a closed configuration. When it is desired to open jaws 177A, 177B, shaft 170 is retracted proximally to expose jaws 177A, 177B such that their natural bias opens jaws 177A, 177B, as shown in FIG. 4B. With the repositioning wire 120B disposed between jaws 177A, 177B, shaft 170 is moved distally to force jaws 177A, 177B together, as shown in FIG. 4C. Snare device 130 may then be rotated in direction R1, as shown in FIG. 4D, to pull the repositioning wire by wrapping it around shaft 170, as explained above. Other clasping mechanisms may be utilized. For example, and not by way of limitation, the jaws may be opening and closed by a mechanical linkage extending proximally to a handle which is operated by the user. Other clasping mechanisms which do not necessarily include two jaws, may also be utilized.

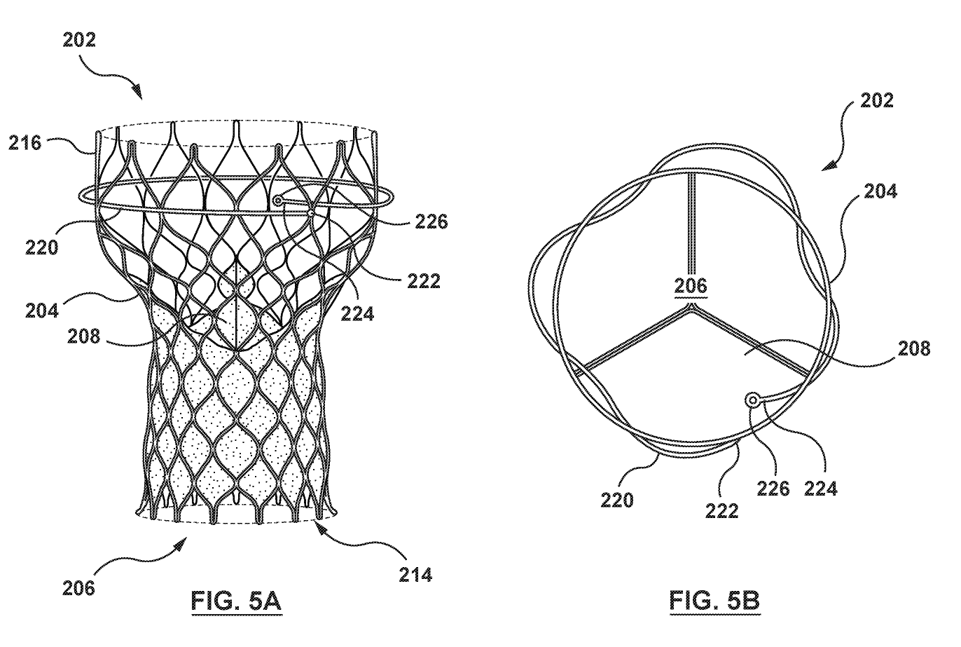

FIGS. 5A-7C illustrate schematically a valve assembly repositioning system 200 including a valve assembly 202 and a snare device 230 according to another embodiment hereof. Valve assembly repositioning system 200 is similar to valve assembly repositioning system 100 described above. In particular, valve assembly 202 is shown in FIGS. 5A-5B and includes a frame 204 and a prosthetic valve 208 as described above. Frame 204 and prosthetic valve 208 may be similar to frame 104 and prosthetic valve 108 describe above and the description in paragraphs [0040]-[0043] above, which are incorporated herein with respect to frame 204 and prosthetic valve 208. Thus, as described above, frame 204 includes a first end 214, a second end 216, and defines a central passage 206 therethrough. Frame 204 includes a number of wire members 210 arranged relative to each other to create open spaces 212. Prosthetic valve 208 is coupled to frame 204 and disposed within central passage of frame 204.

Similar to the embodiments described above, valve assembly further includes a repositioning wire 220. Repositioning wire 220 includes a first end 222 coupled to frame 204. Repositioning wire 220 wraps around a circumference of frame 204 to a second end 224, as shown in FIGS. 5A-5B. With valve assembly 202 in the radially expanded fully deployed configuration, repositioning wire 220 wraps around the circumference of frame 204 at least 75% of the circumference of frame 204 at the location of repositioning wire 220. As previously described, repositioning wire 220 may be woven through open spaces 212 of frame 204, above some frame members 210 and below others.

In the embodiment shown in of FIGS. 5A-7C, a lasso 226 is coupled to second end 224 of repositioning wire 220. Lasso 226 is of a generally circular shape creating a loop. Lasso 226 may be coupled to second end 224 by laser or ultrasonic welding, adhesives, or other methods suitable for the purposes disclosed herein. Alternatively, lasso 226 may be formed as an extension of repositioning wire 220 such that repositioning wire 220 forms a loop which is coupled to the remainder of repositioning wire 220 by a knot, laser or ultrasonic welding, adhesives, or other suitable connection methods. Repositioning wire 220 and lasso 226 may be made of the same materials described above for the repositioning wires.

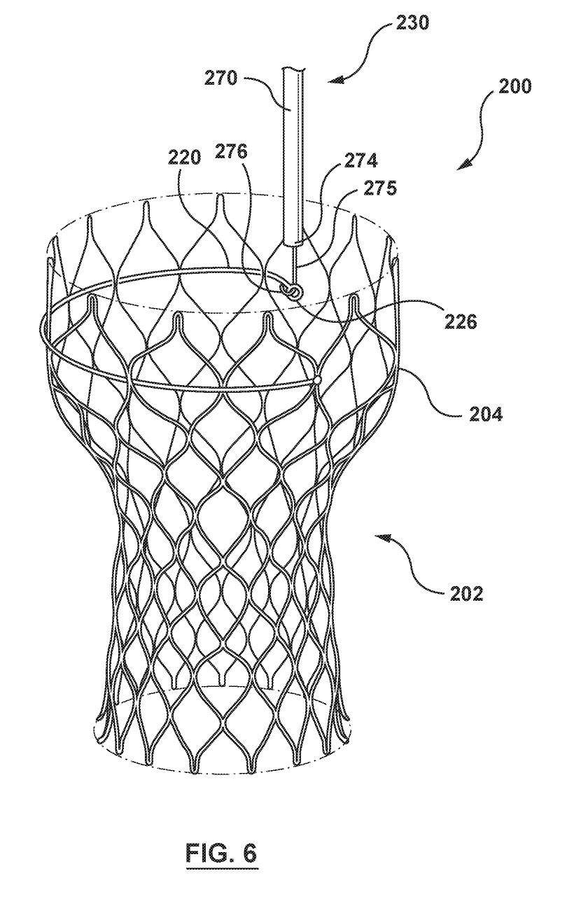

In the embodiment of FIGS. 5A-5B and 6, a single repositioning wire 220 is shown disposed around second end 216 of frame 204. However, as described above, more than one repositioning wire may be utilized. Further, repositioning wire 220 is not limited to the location at the second end 216 of frame 204.

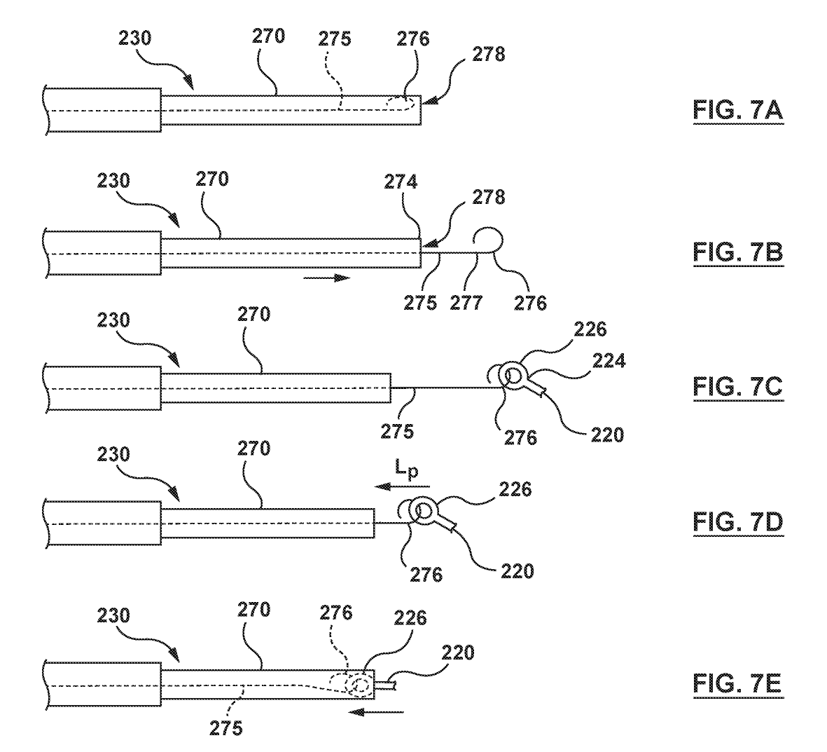

FIGS. 7A-7E show an embodiment of snare device 230. Snare device 230 is an elongated device including a shaft 270 configured for delivery through the vasculature. A snare wire 275 including a snare mechanism 276 at a distal end 277 of snare wire 275 extends through a lumen 278 of shaft 270 and is slidable with respect to shaft 270. In an embodiment, snare mechanism 276 may be a hook, as shown, but other configurations suitable to snaring lasso 226 may also be used. In an embodiment, the position of snare mechanism 276 relative to shaft 270 is user selectable from a location at a proximal end (not shown) of snare device 23. Snare wire 275 and snare mechanism 276 may be constructed of materials such as, but not limited to stainless steel, Nitinol, nylon, polybutester, polypropylene, silk, and polyester or other materials suitable for the purposes described herein.

In an embodiment shown in FIGS. 7A-7E, snare device 230 may be delivered to a location of fully deployed valve assembly 202 with snare mechanism 276 disposed within lumen 278 of shaft 270, as shown in FIG. 7A. Snare device 230 may also be delivered with snare mechanism 276 distal of distal end 274 or snare wire may be delivered through lumen 278 after shaft 270 is delivered. When at the desired location, snare wire 275 is extended distally relative to shaft 270 such that snare mechanism 276 is distal of distal end 274 of shaft 270, as shown in FIG. 7B. Snare mechanism 276 is then manipulated to snare lasso 226 of repositioning wire 220, as shown in FIGS. 6 and 7C. With snare mechanism 276 engaged with lasso 226, snare wire 275 may be retracted proximally in direction Lₚ to pull lasso 226 (and thus repositioning wire 220 coupled to lasso 226) towards shaft 270, as shown in FIG. 7D. As lasso 226 and repositioning wire 220 are pulled, valve assembly 202 is compressed from the radially expanded fully deployed configuration to the radially compressed repositioning configuration. Snare wire 275 may be retracted proximally until snare mechanism 276 and lasso 220 are disposed within lumen 278 of shaft 270, as shown in FIG. 7E.

With snare mechanism 276 engaged with lasso 226 and repositioning wire 220 pulled such that valve assembly 202 is in the radially compressed repositioning configuration, snare device 230 may be manipulated to move valve assembly 202 within the native vessel/valve. To move valve assembly 202 distally or proximally, the user pushes or pulls snare device 230, respectively. Stated another way, when snare device 230 has snared and pulled lasso 226 of repositioning wire 220, and valve assembly 202 is in the radially compressed repositioning configuration, moving snare device 230 distally moves valve assembly 202 distally, and moving snare device 230 proximally moves valve assembly 202 proximally.

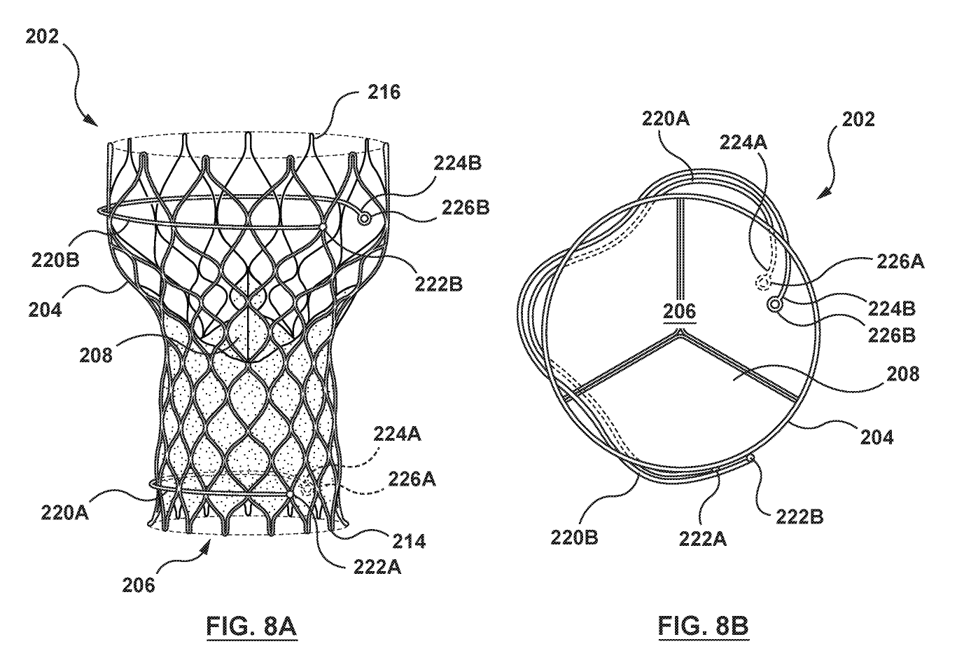

As noted above, valve assembly 202 may include more than one repositioning wire. FIG. 8 shows valve assembly 202 including first and second repositioning wires 220A, 220B at first end 214 and second end 216 of frame 204, respectively. Valve assembly 202 shown in FIG. 8 is the same as the embodiment shown in FIGS. 5A-5B except for the additional repositioning wire. Thus, details of the valve assembly will not be repeated with respect to this embodiment, but the details described above with respect to other embodiments are incorporated herein.

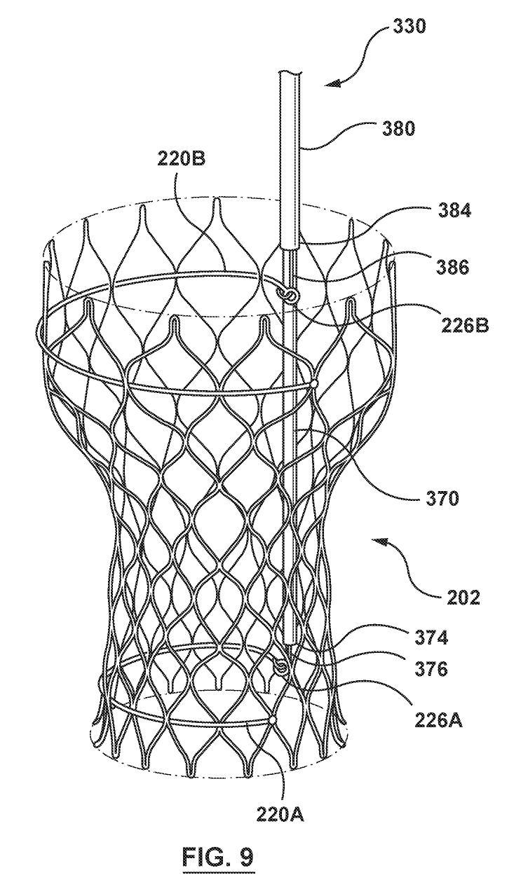

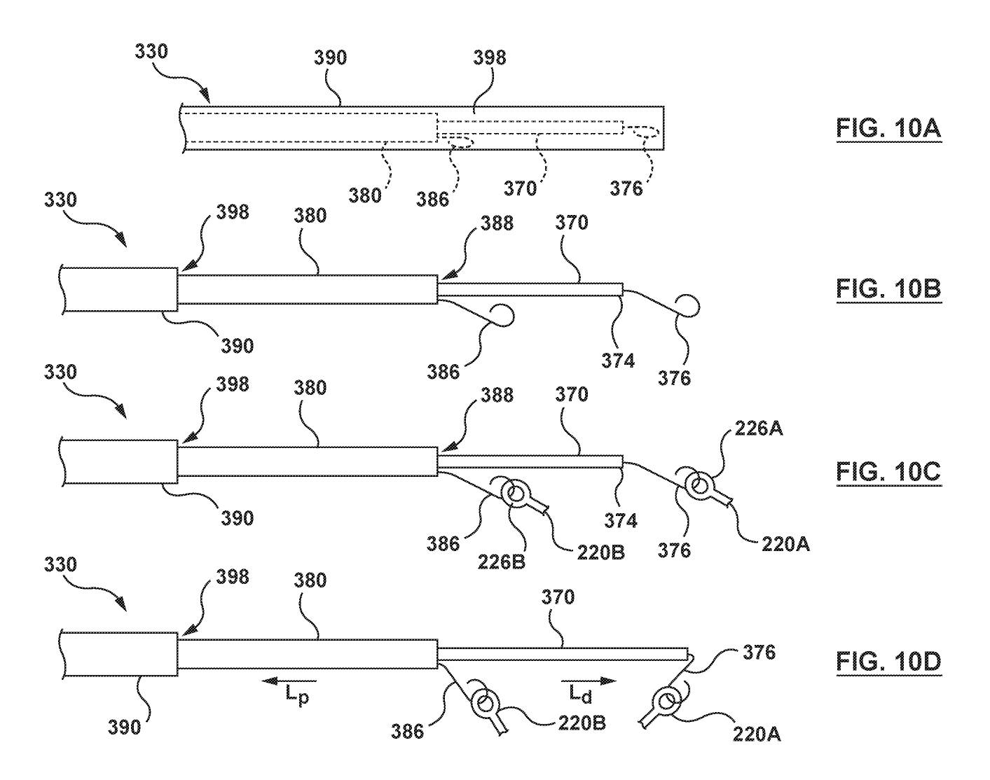

FIGS. 9 and 10A-10D show an embodiment of a snare device 330 which may be used in conjunction with valve assembly 202 including first and second repositioning wires 220A, 220B. Snare device 330 is an elongated device including a first snare 376 and a second snare 386. First snare 376 is disposed at a distal end 374 of a first shaft or wire 370. Second snare 386 is disposed at a distal end 384 of a second shaft 380. Second shaft 380 defines a lumen 388 through which first shaft 370 is slidably disposed. Snare device 330 may optionally include a third shaft 390 including a lumen 398. First and second shafts 370, 380 are slidable through third shaft 390. The position of first snare 376 relative to second snare 386 of snare device 330 is user adjustable by sliding first shaft 370 and second shaft 380 relative to each other. First snare 376 and second snare 386 are curved or sharply bent shapes suitable for catching, snagging, or snaring first lasso 226A and second lasso 226B, respectively. While FIGS. 9 and 10A-10D show first snare 376 and second snare 386 as a hook shape, this is not meant to limit the design and other shapes or constructions may be provided that are suitable for the purposes outlined herein. First and second snares 376, 386 may be constructed of materials such as, but not limited to stainless steel, Nitinol, nylon, polybutester, polypropylene, silk, and polyester or other materials suitable for the purposes described herein.

Snare device 330 may be advanced to a location of a fully deployed valve assembly in the configuration shown in FIG. 10A, with first and second shafts 370, 380 and first and second snares 376, 386 disposed within lumen 398 of third shaft 390. However, snare device 330 may be advanced to the location of a fully deployed valve assembly in other configurations. When at the desired location, first snare 376 and second snare 386 are exposed, for example, by retracting third shaft 390 or advancing first and second shafts 370, 380, as shown in FIG. 10B. First snare 376 and second snare 386 are manipulated such that first snare 376 is placed through first lasso 226A of repositioning wire 220A and second snare 386 is placed through second lasso 226B of repositioning wire 220B, as shown in FIG. 10C.

With first snare 376 engaged with first lasso 226A and second snare 386 engaged with second lasso 226B, first and second shafts 376, 386 may be moved in opposite directions, as indication by arrows L_{d} and Lₚ, respectively, in FIG. 10D. In the embodiment shown, first snare 376 is move distally and second snare 386 is moved proximally. Moving first snare 376 and second snare 386 in opposite directions causes first lasso 226A and second lasso 226B to be pulled, thereby causing first repositioning wire 220A and second repositioning wire 220B to be pulled, respectively. Pulling of first repositioning wire 220A and second repositioning wire 220B radially compresses valve assembly 202 from the radially expanded fully deployed configuration to the radially compressed repositioning configuration, as explained above.

With first and second snares 376, 386 engaged with first and second lassos 226A, 226B and first and second repositioning wires 220A, 220B pulled such that valve assembly 202 is in the radially compressed repositioning configuration, snare device 330 may be manipulated to move valve assembly 202 within the native vessel/valve. To move valve assembly 202 distally or proximally within the native valve, the user moves snare device 330 distally or proximally, respectively. Stated another way, when snare device 330 has snared and pulled first and second lassos 226A, 226B of first and second repositioning wires 220A, 220B, respectively, and valve assembly 202 is in the radially compressed repositioning configuration, moving snare device 330 distally moves valve assembly 202 distally, and moving snare device 330 proximally moves valve assembly 202 proximally.

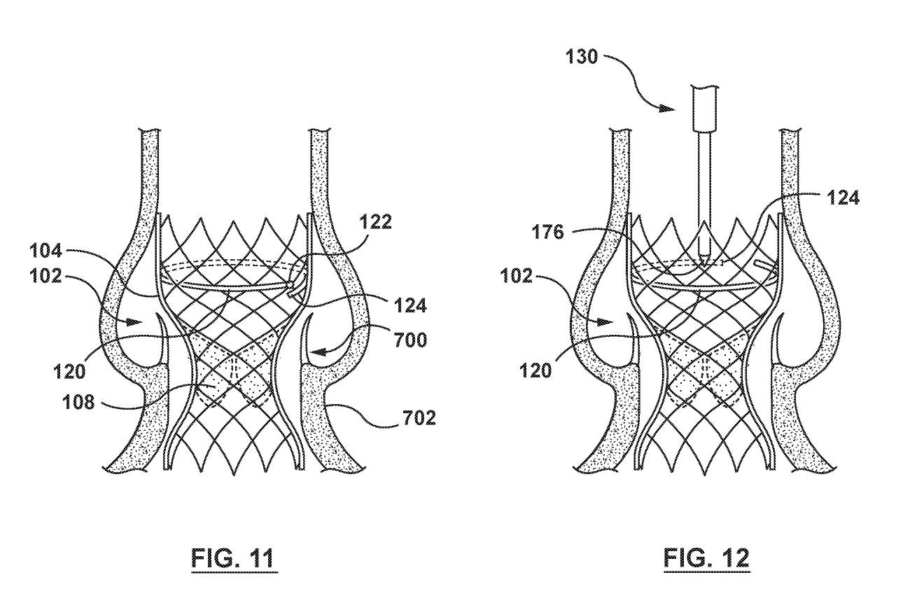

An embodiment of a method of repositioning a fully deployed valve assembly in a native valve is schematically represented in FIGS. 11-16. Although the method is described with respect to valve assembly 102 and snare device 130, it will be apparent to one of ordinary skill that methods described herein may be utilized with valve assemblies and snare devices according to any embodiment described herein. In FIG. 11, a valve assembly 102 is fully deployed adjacent a native valve 700 at a first location. Valve assembly 102 is in a radially expanded fully deployed configuration and is thus disconnected from a delivery device. As previously described, valve assembly 102 includes a frame 104, a repositioning wire 120, and a prosthetic valve 108. As determined by the treating clinician, valve assembly 102 may not be performing as desired, and repositioning of valve assembly 102 is desired to improve valve performance. For example, and not by way of limitation, it may be determined that valve assembly 102 is “too deep” into the annulus 702 such that it may interfere with the left ventricular outflow tract (LVOT) or form gaps between frame 104 and annulus 702 causing paravalvular leakage. In another example, valve assembly 102 may not be deep enough in annulus such that frame 104 is not properly secured against annulus 702, which may also cause paravalvular leakage.

Snare device 130 is advanced through the patient's vasculature and is positioned adjacent valve assembly 102. Clasping mechanism 176 of snare device 130 is manipulated by the treating clinician to grasp a second end 124 of repositioning wire 120, as shown in FIG. 12.

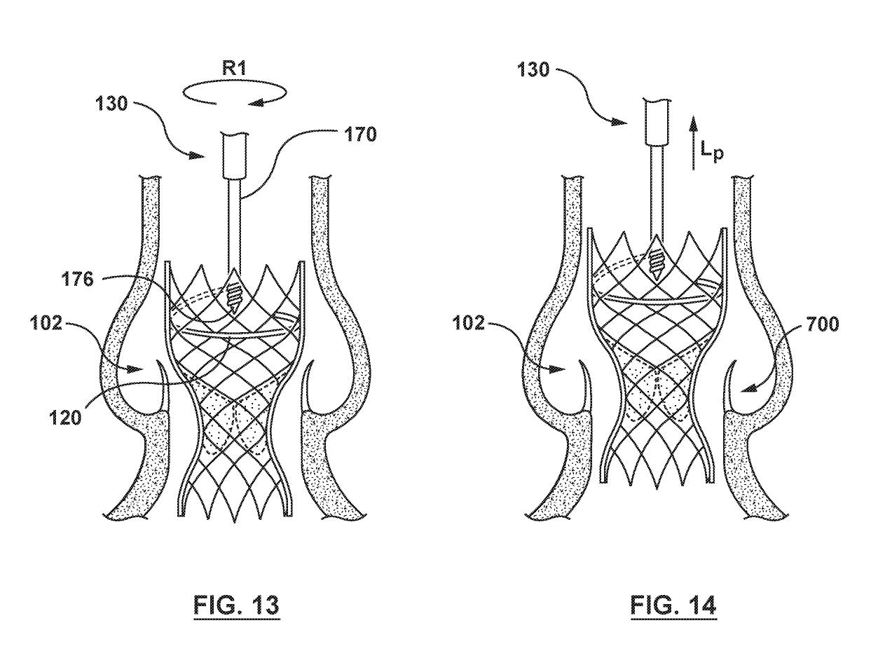

Snare device 130 is rotated in a direction R1 by the treating clinician, thereby pulling repositioning wire 120 as it wraps circumferentially around shaft 170 of snare device 130. The pulling of repositioning wire 120 compresses valve assembly 102 from the radially expanded fully deployed configuration to the radially compressed repositioning configuration, as shown in FIG. 13.

Snare device 130 may then be moved proximally or distally to reposition valve assembly 102 from the first location of FIG. 11 to a second location adjacent native valve 700. FIG. 14 shows snare device 130 and valve assembly 102 being moved proximally in a direction Lₚ to the second location as determined by the treating clinician. However, valve assembly 102 may be moved distally.

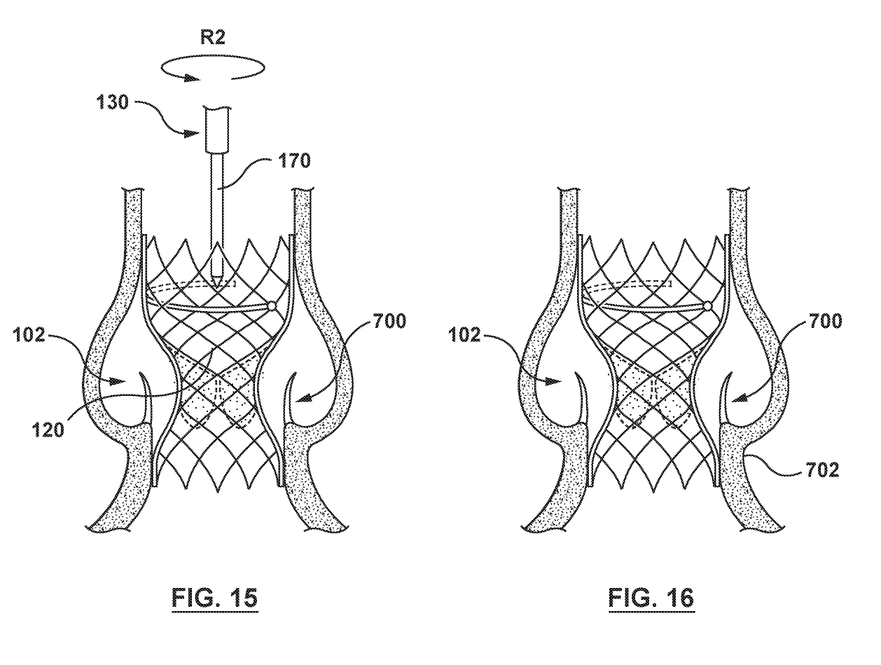

Snare device 130 is rotated in a direction R2 opposite direction R1 by the treating clinician, thereby releasing the pulling force on repositioning wire 120 as repositioning wire 120 unwraps circumferentially from shaft 170 of snare device 130. As the pulling force is released, frame 104 of valve assembly 102 self-expands from the radially compressed repositioning configuration to the radially expanded fully deployed configuration at the second location, as shown in FIG. 15.

Once valve assembly 102 is in its radially expanded fully deployed configuration at the second location, snare device 130 may be withdrawn from the patient. Valve assembly 102 remains fully deployed at the repositioned second location adjacent native valve 700, as shown in FIG. 16.

A similar method may be used for a valve assembly 102 including a plurality of repositioning wires 120. For example, and not by way of limitation, a snare device may be used for each repositioning wire. In another example, a snare device may include multiple clasping mechanisms, one for each of the plurality of repositioning wires. Further, a similar method may be used to reposition valve assembly 202 of FIGS. 5A-5B utilizing the snare device of FIGS. 7A-7E.

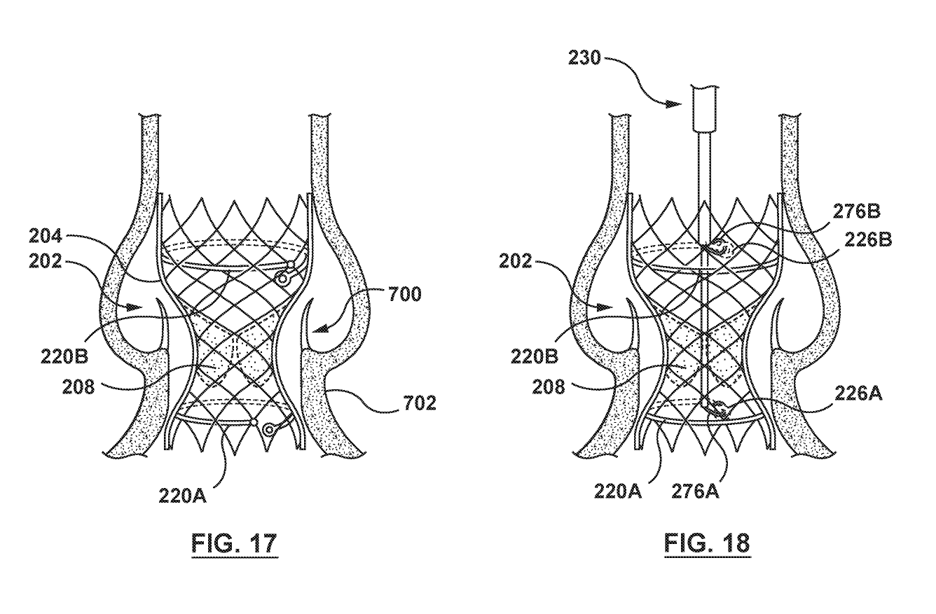

FIGS. 17-22 schematically show a method of repositioning a fully deployed valve assembly 202 in accordance with another embodiment hereof. Although described herein with respect to valve assembly 202 of FIGS. 8A-8B and snare device 330 of FIGS. 10A-10D, it will be apparent to one of ordinary skill that methods described herein may be used with valve assemblies and snare devices according to any embodiment described herein. In FIG. 17, a valve assembly 202 is fully deployed adjacent a native valve 700 at a first location. Valve assembly 202 is in a radially expanded fully deployed configuration and thus is disconnected from a delivery device. As previously described, valve assembly 202 includes a frame 204, first and second repositioning wires 220A, 220B, and a prosthetic valve 208. As determined by the treating clinician, valve assembly 202 may not be performing as desired, and repositioning of valve assembly 202 is desired to improve valve performance. For example, and not by way of limitation, it may be determined that valve assembly 202 is “too deep” into the annulus 702 such that it may interfere with the left ventricular outflow tract (LVOT) or form gaps between frame 204 and annulus 702 causing paravalvular leakage. In another example, valve assembly 202 may not be deep enough in annulus such that frame 204 is not properly secured against annulus 702, which may also cause paravalvular leakage.

Snare device 330 is advanced through the patient's and is positioned within valve assembly 202, as shown in FIG. 18.

First snare 376 is manipulated by the treating clinician to snare first lasso 226A of first repositioning wire 220A and second snare 386 is manipulated by the treating clinician to snare second lasso 226B of second repositioning wire 220B, as shown in FIG. 18.

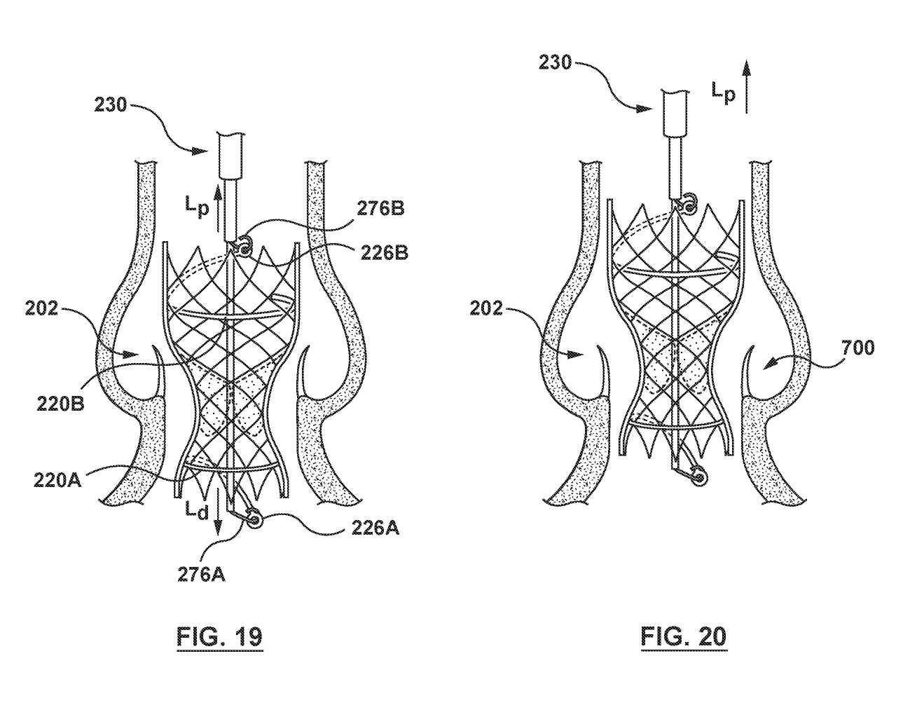

Once first lasso 226A and second lasso 226B are snared by first snare 376 and second snare 386, respectively, snare device 330 is actuated by the treating clinician such that first snare 376 and second snare 386 are moved apart from each other. This movement causes first repositioning wire 220A and second repositioning wire 220B to be pulled in directions L_{d} and Lₚ, respectively, thereby compressing valve assembly 202 from the radially expanded fully deployed configuration to a radially compressed repositioning configuration, as shown in FIG. 19.

Snare device 330 may then be moved proximally or distally to reposition valve assembly 202 from the first location of FIG. 17 to a second location adjacent the native valve 700. FIG. 20 shows snare device 330 and valve assembly 202 being moved proximally in a direction Lₚ to the second location as determined by the treating clinician. However, valve assembly 202 may instead be moved distally.

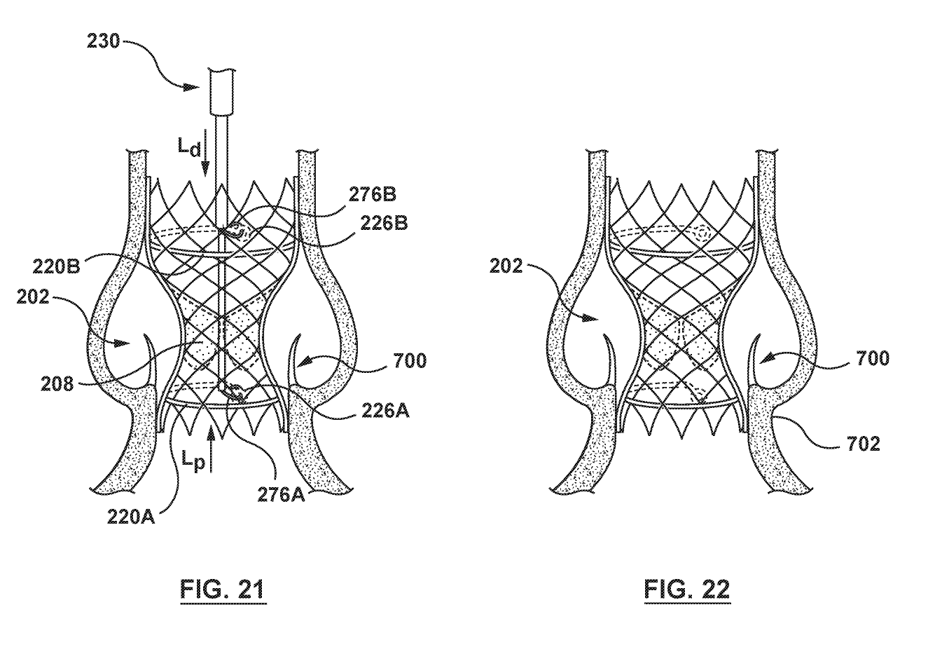

When valve assembly 202 is positioned at the repositioned second location, the treating clinician may move first snare 376 and second snare 386 back toward each other such that the pulling force on first repositioning wire 220A and second repositioning wire 220B is released. As the pulling force on repositioning wires 220A and 220B is released, valve assembly 202 self-expands from the radially compressed repositioning configuration to the radially expanded fully deployed configuration at the second location, as shown in FIG. 21.

Once valve assembly 202 is and in its radially expanded fully deployed configuration at the second location, snare device 330 may be withdrawn from the patient. Valve assembly 202 remains fully deployed at the repositioned second location adjacent native valve 700, as shown in FIG. 22.

While only some embodiments and methods have been described herein, it should be understood that it has been presented by way of illustration and example only, and not limitation. Various changes in form and detail can be made therein without departing from the spirit and scope of the invention, and each feature of each embodiment discussed herein, and of each reference cited herein, can be used in combination with the features of any other embodiment. All patents and publications discussed herein are incorporated by reference herein in their entirety.

What is claimed is:

1. A valve assembly having a radially expanded fully deployed configuration and a radially compressed repositioning configuration, the valve assembly comprising:
a generally tubular frame defining a central passage;
a prosthetic valve coupled to the frame and disposed in the central passage; and
a repositioning wire coupled to the frame and configured such that pulling the repositioning wire radially compresses the valve assembly from the radially expanded fully deployed configuration to the radially compressed repositioning configuration.

2. The valve assembly of claim 1, wherein the frame includes a plurality of frame members with open spaces between the frame members, wherein the repositioning wire is woven through the open spaces, above some frame members, and below other frame members.

3. The valve assembly of claim 1, wherein the repositioning wire comprises a plurality of repositioning wires.

4. The valve assembly of claim 1, wherein the repositioning wire has a first end coupled to the frame, wherein the repositioning wire extends from the first end around at least a portion of a circumference of the frame to a second end of the repositioning wire.

5. The valve assembly of claim 4, wherein with the valve assembly in the radially expanded fully deployed configuration, the repositioning wire extends around at least 75 percent of the circumference of the frame.

6. The valve assembly of claim 4, wherein the second end of the repositioning wire includes a lasso.

7. The valve assembly of claim 1, wherein a diameter of the frame in the radially compressed repositioning configuration is in the range of 40-80 percent of a diameter of the frame in the radially expanded fully deployed configuration.

8. A valve assembly repositioning system comprising:
a valve assembly having a radially expanded fully deployed configuration and a radially compressed repositioning configuration, the valve assembly including a generally tubular frame defining a central passage, a prosthetic valve coupled to the frame, and a repositioning wire coupled to the frame; and
a snare device configured to snare the repositioning wire and to pull the repositioning wire to radially compress the valve assembly from the radially expanded fully deployed configuration to the radially compressed repositioning configuration, wherein the snare device is further configured to move the valve assembly when the valve assembly is in the radially compressed repositioning configuration.

9. The valve assembly repositioning system of claim 8, wherein the repositioning wire comprises a plurality of repositioning wires.

10. The valve assembly repositioning system of claim 8, wherein the repositioning wire has a first end coupled to the frame, wherein the repositioning wire extends from the first end around at least a portion of a circumference of the frame to a second end of the repositioning wire.

11. The valve assembly of claim 10, wherein with the valve assembly in the radially expanded fully deployed configuration, the repositioning wire extends around at least 75 percent of the circumference of the frame.

12. The valve assembly repositioning system of claim 8, wherein the snare device includes a clasping mechanism at a distal second end of the snare device.

13. The valve assembly repositioning system of claim 8, wherein the repositioning wire includes a lasso.

14. The valve assembly repositioning system of claim 8,
wherein the repositioning wire includes a first repositioning wire disposed adjacent a first end of the frame and including a first lasso, and a second repositioning wire disposed adjacent a second end of the frame and including a second lasso,
wherein the snare device includes a first snare configured to snare the first lasso and a second snare configured to snare the second lasso, and
wherein the snare device is configured such that the first snare and the second snare are moved apart from each other to move the first lasso and the second lasso apart from each other to radially compress the valve assembly.

15. The valve assembly repositioning system of claim 14, wherein the first snare is disposed at a distal end of a first shaft and the second snare is disposed at a distal end of a second shaft, wherein the first shaft is disposed within a lumen of and is slidable relative to the second shaft.

16. The valve assembly of claim 8, wherein a diameter of the frame in the radially compressed repositioning configuration is in the range of 40-80 percent of a diameter of the frame in the radially expanded fully deployed configuration.

17. A method of repositioning a valve assembly, the valve assembly including a tubular frame, a prosthetic valve coupled to the frame, and a repositioning wire coupled to the frame, the method comprising the steps of:
advancing a snare device to a location of the valve assembly with the valve assembly being in a radially expanded fully deployed configuration at a first location adjacent a native valve;
snaring the repositioning wire with the snare device;
manipulating the snare device such that the repositioning wire is pulled to radially compress the valve assembly from the radially expanded fully deployed configuration to a radially compressed repositioning configuration;
moving the snare device to move the valve assembly from the first location to a second location adjacent the native valve; and
releasing the repositioning wire from the snare device to radially expand the valve assembly from the radially compressed repositioning configuration to the radially expanded fully deployed configuration.

18. The method of claim 17, wherein the repositioning wire includes a first end coupled to the frame and the reposition wire extends from the first end at least partially around a circumference of the frame to a second end of the repositioning wire, wherein the snare device includes a clasping mechanism, and wherein the step of snaring the repositioning wire comprises clasping the second end of the repositioning wire with the clasping mechanism and wherein the step of releasing the repositioning wire comprises unclasping the second end of the repositioning wire from the clasping mechanism of the snare device.

19. The method of claim 18, wherein the step of manipulating the snare device comprises rotating the snare device in a first direction such that the repositioning wire wraps circumferentially around a shaft of the snare device, and wherein the step of releasing the repositioning wire comprises rotating the snare device in a second direction opposite of the first direction such that the repositioning wire unwraps circumferentially from the shaft of the snare device prior to unclasping the clasping mechanism.

20. The method of claim 17, wherein the repositioning wire includes a lasso, and the snare device includes a snaring mechanism, wherein the step of snaring the repositioning wire comprises snaring the lasso with the snaring mechanism, and wherein the step of releasing the repositioning wire comprises releasing the lasso from the snaring mechanism.

21. The method of claim 17, wherein the repositioning wire includes a first repositioning wire disposed adjacent a first end of the frame and including a first lasso, and a second repositioning wire disposed adjacent a second end of the frame and including a second lasso, and
wherein the snare device includes a first snare configured to snare the first lasso and a second snare configured to snare the second lasso,
wherein the step of snaring the repositioning wire comprises the first snare snaring the first lasso and the second snare snaring the second lasso, and
wherein the step of manipulating the snare device comprises moving the first snare and the second snare apart from each other to move the first lasso and the second lasso apart from each other to radially compress the valve assembly.

22. The method of claim 21, wherein the first snare is disposed at a distal end of a first shaft and the second snare is disposed at a distal end of a second shaft, wherein the first shaft is disposed within a lumen of and is slidable relative to the second shaft, wherein the step of moving the first snare and the second snare apart from each other comprises sliding the first shaft and the second shaft relative to each other.
